(19) United States
(12) Patent Application Publication
(10) Pub. No.: US 2017/0340986 A1
(43) Pub. Date: Nov. 30, 2017

(54) DISTILLATION PROCESS COMPRISING AT LEAST TWO DISTILLATION STEPS TO OBTAIN PURIFIED HALOGENATED CARBOXYLIC ACID HALIDE, AND USE OF THE PURIFIED HALOGENATED CARBOXYLIC ACID HALIDE

(71) Applicant: SOLVAY SA, Brussels (BE)

(72) Inventor: Max Josef BRAUN, Wedemark (DE)

(21) Appl. No.: 15/527,436

(22) PCT Filed: Nov. 17, 2015
(86) PCT No.: PCT/EP2015/076838

§ 371 Date: May 17, 2017

(30) Foreign Application Priority Data
Nov. 17, 2014 (EP) 14193476.0

(51) Int. Cl. B01D 3/14; C07C 53/48; C07C 51/64

(52) U.S. Cl. CPC B01D 3/143; C07C 51/64; C07C 53/48

(57) ABSTRACT

The present invention concerns a process for the production of a fluorinated carboxylic halide having a reduced content of impurities, a fraction of the fluorinated carboxylic halide having a reduced content of impurities, and its use in the manufacture of agriculturally and pharmaceutically active compounds or their intermediates.

The present invention concerns a process for the production of a fluorinated carboxylic halide having a reduced content of impurities, a fraction of the fluorinated carboxylic halide having a reduced content of impurities, and its use in the manufacture of agriculturally and pharmaceutically active compounds or their intermediates.

Trifluoroacetylchloride (TFAC), difluoroacetylchloride (DFAC) or chlorodifluoroacetylchloride (CDFAC), are valuable intermediates in chemical synthesis, for example in the preparation of herbicides, surfactants and pharmaceuticals. For example, trifluoroacetyl chloride is a starting material for the synthesis of 4-ethoxy-1,1,1-trifluoromethyl-3-buten-2-one, which can suitably be converted into cyclic intermediates for agriculturally active ingredients, see, for example WO2011/3860 and WO2010037688. CDFAC can, for example, be converted to fluorosubstituted-3-oxo-alcanoic acids, which can further be converted into intermediates for agriculturally active compounds, see for example WO2010037688 and WO2012/25469. DFAC is, for example, used for the synthesis of CDK inhibitors, as described in WO2006/64251, or agriculturally active compounds, as described, for example, in WO2005/42468.

CN103524325 describes the purification of a fraction containing 1,1-difluoroacetyl chloride by compression distillation.

In particular, for industrial manufacture of building blocks for agriculturally and pharmaceutically active compounds, the purity of fluorinated carboxylic acid halides is critical for the quality of downstream products, viability of apparatus, in particular in view of corrosive impurities, and waste management. There is an ongoing industrial need for a scalable process for the purification of fluorinated carboxylic halides.

In consequence, the invention concerns a process for producing a compound of the formula (I) R1-C(O)X having a reduced content of impurities, wherein R1 is CF₂H, CF₃ or CClF2 and X is a halogen, which comprises a) subjecting a crude fraction comprising compound of formula (I) R1-C(O)X and impurities to at least two distillation steps, wherein the at least two distillation steps are performed at different temperatures and b) recovering at least a fraction of the compound of the formula (I) having a significantly reduced content of impurities. The invention further concerns a fraction of a compound of formula (I) R1-C(O)X having a reduced content of impurities, obtainable by the said distillation process, in particular when the compound of formula (I) was manufactured by oxidation of a compound of formula (II) R1-CHX′₂, and the use of the fraction having a reduced content of impurities for the manufacture of a pharmaceutically or agriculturally active compounds or their intermediates.

Another aspect of the present invention is a process for the manufacture of agriculturally or pharmaceutically active compounds, comprising the process for the production of a compound of the formula (I) R1-C(O)X having a reduced content of impurities.

It has been found that a compound of the formula (I) R1-C(O)X, in particular CDFAC, having a reduced content of impurities can advantageously be obtained by applying a distillation process comprising at least two, preferably at least three distillation steps at different temperatures. The process makes it possible to achieve an efficient separation of impurities from the compound of formula (I) R1-C(O)X, in particular CDFAC, by a physical method. The recovered purified fraction of (I) R1-C(O)X can be used as starting material for the lab scale industrial scale synthesis and manufacture of further compounds and building blocks, in particular for agriculturally or pharmaceutically active compounds, while having a reduced amount of impurities, which allows for reduced corrosion in apparatus and a reduced amount of impurities and waste in downstream processes. The process effectively reduces both inorganic, for example hydrogen halides, and organic impurities. Especially if phosgene is present, the purification process also allows for the waste management of said phosgene as a fraction phosgene and often the compound of formula (I) R1-C(O)X by recycling said fraction into the process by which the crude fraction containing the compound of the formula (I) R1-C(O)X is obtained, in particular if said process is the oxychlorination of alkanes or olefins. The process according to the present invention can be carried out in an easy manner and allows for use of distillation apparatus made or partially made or coated or partially coated with glass or enamel.

In the process according to the present invention, R1 is CClF₂, CF₂H or CF₃, wherein CClF₂ is preferred. X is selected from the group consisting of Cl, F and Br, wherein Cl is preferred. Most preferably, the compound of formula (I) is chlorodifluoro-acetyl chloride (CDFAC), wherein R1 is CClF2 and X is chlorine.

Acid halides used in the present invention can be obtained, for example, by photooxidation of halogenated precursor alkanes, in particular as described in U.S. Pat. No. 5,569,782 the content of which is incorporated by reference in the present application. In particular, chlorodifluoro acetyl chloride, which is a particularly preferred compound of formula (I) in the present invention, can be obtained by photooxidation of 1-chloro-1,1-difluoro-2,2-dichloroethane (R 122). Other ways to manufacture acid halides of formula (I) are described, for example, in EP0623577, U.S. Pat. No. 5,545,298A, U.S. Pat. No. 4,643,851, U.S. Pat. No. 5,241,113, U.S. Pat. No. 5659078, U.S. Pat. No. 6,255,524 and U.S. Pat. No. 7,754,927. Generally, the purification method according to the present invention is suitable for reducing impurities in crude fractions containing a compound of formula (I) and impurities, regardless of the way how the compound according to formula (I) was produced. The manufacture of the fraction containing the compound of formula (I) and impurities by oxidation of formula (II) R1-CHX′₂, wherein R1 has the same definition as above, and X′ is the same or different, wherein X′ is a halogen selected from the group consisting of Cl, F and Br, in particular wherein X′ is Cl, is particularly preferred in view of the effectiveness of the purification process according to the present invention.

According to the present invention, a crude fraction comprising the compound of formula (I) R1-C(O)X, in particular CDFAC, and impurities is subjected to at least two, preferably three distillation steps which are performed at different temperatures. When two distillation steps are applied, these consist of a high temperature distillation step and a low temperature distillation step. In a preferred embodiment, a) comprises at least three distillation steps, which consist of a high temperature distillation step, a medium temperature distillation step and a low temperature distillation step. According to a preferred embodiment, the low temperature distillation step is performed first, the medium temperature distillation step is performed second and the high temperature distillation step is performed third. Generally, the at least two, preferably least three distillation steps can be performed in an order which is suited for the impurity profile of the crude fraction in order to obtain a fraction containing compound (I) and a reduced amount of impurities. Preferably, the fraction of the compound of formula (I), in particular CDFAC, having a reduced content of impurities is recovered from the high temperature distillation step as bottom product.

In the present description, any reference to the temperature corresponds to the temperature measured at the top of the distillation column, also indicated as “head temperature”. The temperature of the bottom of the distillation is adjusted accordingly.

In a preferred embodiment, at least three distillation steps are applied in a). Preferably, the at least three distillation steps consist of a low temperature distillation step, a medium temperature distillation step and a high temperature distillation step. Concerning the temperature values which are applied in the different distillation steps, the medium temperature distillation step is generally carried out at a pressure of at least 0.5° C. lower than the high temperature distillation step. Generally, the pressure difference between the high temperature distillation step and the medium temperature distillation step is from 0.5 to 15° C. Often, the temperature difference between the high temperature distillation step and the medium temperature distillation step is equal to or more than 1° C., preferably equal to or more than 2° C. and most preferably equal to or more than 3° C. Often, the temperature difference between the high temperature distillation step and the medium temperature distillation step is equal to or less than 15° C., preferably equal to or less than 14° C. and most preferably equal to or less than 13° C. Generally, the low temperature distillation step is generally carried out at a pressure of at least 0.5° C. lower than the medium temperature distillation step.

Generally, the pressure difference between the low temperature distillation step and the medium temperature distillation step is from 0.5 to 15° C. Often, the temperature difference between the low temperature distillation step and the medium temperature distillation step is equal to or more than 1° C., preferably equal to or more than 2° C. and most preferably equal to or more than 3° C.

Often, the temperature difference between the low temperature distillation step and the medium temperature distillation step is equal to or less than 15° C., preferably equal to or less than 14° C. and most preferably equal to or less than 13° C. Generally, the medium temperature distillation step is carried out at a head temperature of at least 5° C. lower than the high temperature distillation step, and the low temperature distillation step carried out at a head temperature of at least 5° C. lower than the medium temperature distillation step.

When three distillation steps are applied in a), generally, the temperature in the high temperature distillation step is from −8 to 8° C. Often, the temperature in the high temperature distillation step is equal to or more than −5° C., preferably equal to or more than −3° C. and most preferably equal to or more than −1° C.

Often, the temperature in the high temperature distillation step is equal to or lower than 5° C., preferably equal to or lower than 3° C. and most preferably equal to or lower than 1° C.

When three distillation steps are applied in a), generally, the temperature in the medium temperature distillation step is from −18 to −2° C. Often, the temperature in the medium temperature distillation step is equal to or more than −15° C., preferably equal to or more than −13° C. and most preferably equal to or more than −11° C. Often, the temperature in the high temperature distillation step is equal to or lower than −5° C., preferably equal to or lower than −7° C. and most preferably equal to or lower than −8° C.

When three distillation steps are applied in a), generally the temperature in the low temperature distillation step is from −28 to −12° C. Often, the temperature in the low temperature distillation step is equal to or more than −25 ° C., preferably equal to or more than −23° C. and most preferably equal to or more than −21° C. Often, the temperature in the low temperature distillation step is equal to or lower than −15° C., preferably equal to or lower than −17° C. and most preferably equal to or lower than −19° C.

The fraction of the compound of formula (I) having a reduced content of impurities is preferably recovered from the high temperature distillation step as bottom product.

In a preferred embodiment, the compound of formula (I) is CDFAC, and the distillation is carried out in a first, low temperature distillation step at a temperature from −22 to −18° C., then in a second, medium temperature distillation step at a temperature from −12 to −8° C. and then in a third, high temperature distillation step at a temperature from −2 to 2° C. This is particularly preferred if the crude fraction containing CDFAC and impurities, which is fed to the distillation process, was obtained by photooxidation of 1-chloro-1,1-difluoro-2,2-dichloroethane (R122). In this embodiment, the fraction A obtained at the distillation top of the first, low temperature distillation step comprises COF2, HCl and Cl2 and is often fed into a scrubber system. In another aspect, this fraction A may further be used as crude product for other processes. The bottom fraction, often comprising CDFAC, CF2Cl-CHCl2 (R 122), COC12 and 1,1difluorotetrachloroethane (R 112a), of this first, low temperature, distillation step is fed to the medium temperature distillation step. The fraction B obtained at the distillation column top of the second, medium temperature distillation step comprises COC12 and CDFAC and is fed to the third, high temperature distillation step. The bottom fraction of the second, medium temperature distillation step often comprises CF2C1-CHC12 (R 122) and 1,1difluorotetrachloroethane (R 112a) and can be recycled to the process by which the crude fraction containing the compound of formula (I) was obtained, or can further be used as crude product for other processes. The fraction C obtained at the distillation column top of the third, high temperature distillation step comprises COC12 and a low content of CDFAC. Fraction C can preferably be fed into the process to the process by which the crude fraction containing the compound of formula (I) was obtained, in particular if said process is a photooxidation process. The COC12 contained in fraction C there is suitably oxidized to CO2, thus handling and waste management of the hazardous COC12 is avoided. CDFAC contained in fraction C advantageously accumulates in further distillation steps. The bottom fraction of the third distillation step is recovered as CDFAC having a reduced content of impurities, preferably as fluid.

Preferably, the process for the production of a compound of the formula (I) R1-C(O)X having a reduced content of impurities is carried out at a pressure of 0.7 to 1.3 bar. Preferably, the process is carried out at ambient pressure, ambient pressure denoting the pressure which is given by the natural conditions of the environment of the distillation apparatus. Often, the pressure is about 1 bar.

Generally, it is possible to feed the fractions to be distilled to the distillation column at any location of the distillation column, be it at the top, the bottom, or anywhere between the bottom and the top. Often, it is preferred to feed the fraction to be distilled to the distillation column at a location of at least 20% of the theoretical plates, measured from the bottom of the column, more preferably at a location of at least 30% of the theoretical plates, measured from the bottom of the column, and even more preferably at a location of at least 40% theoretical plates, measured from the bottom of the column. A most preferred location is of at least 50% of the theoretical plates, measured from the bottom of the column.

The distillation columns which can be used in the process according to the invention are known per se. Most preferred are columns made of or partially made of or coated with or partially coated with glass or enamel. Often, the columns are conventional plate columns or plate columns of dual-flow type or alternatively of columns with bulk or structured packing. Glass or enamel packing is preferred.

In one embodiment of the present invention, the crude fraction of the compound of formula (I) has been obtained by an oxidation process starting from a compound of formula (II) R1-CHX′₂, wherein X′ is the same or different, wherein X′ is a halogen selected from the group consisting of Cl, F and Br, in particular wherein X′ is Cl, and wherein R1 has the same definition as above. In one preferred aspect of this embodiment, the oxidation process is a photooxidation process in the presence of oxygen, in particular wherein said photooxidation is further carried out in the presence of added elemental chlorine. A photooxidation according to this embodiment is particularly advantageous when a Hg high-pressure lamp doped with a metal iodide is used as a source for the activating radiation, in particular wherein the metal iodide is selected from the group consisting of gallium iodide, thallium iodide or cadmium iodide. Details of such a process are described in EP0638539A, in particular with respect to the oxidation process by which the crude fraction CDFAC is obtained from CF₂Cl—CHCl₂ (R 122), which is incorporated hereby in its entirety.

Generally, the process for producing a compound of the formula (I) R1-C(O)X, in particular CDFAC, having a reduced content of impurities can be carried out batch-wise or continuous. In a batch-wise process, the process can be carried out in a distillation apparatus which comprises one or more distillation columns, wherein one distillation column is preferred. In a continuous process, the process can be carried out in a distillation apparatus which comprises two or more distillation columns, wherein three or more distillation column are preferred; three distillation columns are most preferred. The continuous process is preferred.

In one aspect of the present invention, a fraction comprising the compound of formula (I), in particular CDFAC, and at least one impurity, in particular COCl₂, is recovered from the high temperature distillation step as top product, and wherein the fraction comprising the compound of formula (I) and at least one impurity is fed into a previous process step of the process for the manufacture of the crude fraction of the compound of formula (I) which is fed to the process for the production of a compound of the formula (I) R1-C(O)X having a reduced content of impurities. As described above, the COCl2 contained in the fraction obtained as top product from a third, high temperature distillation step, also denoted as fraction C, there is often suitably oxidized to CO2,thus handling and waste management of the hazardous COCl₂ is avoided. CDFAC contained in fraction C advantageously accumulates in further distillation steps.

It was found that in the process according to the present invention, a purified fraction of the compound of formula (I) can suitably be obtained by an industrially applicable distillation process by applying at least two preferably three distillation steps at different temperatures. The process not only allows for efficient separation and recovery of fractions that a purified and/or can suitably be disposed of and/or recyled. The process also allows for a distillation procedure under ambient pressure, which is unexpected as the state of the art describes compressed distillation procedures or non-industrial distillation procedures not employing at least two steps, in which the advantageous results cannot be obtained, or where compression distillation has to be applied, which is less benificial economically and may also yield less advantageous chemical results.

The invention further concerns a fraction of a compound of formula (I) R1-C(O)X, in particular CDFAC, having a reduced content of impurities, obtainable by the process according to the present invention. This is particularly preferred if fraction of CDFAC having a reduced content of impurities was obtained by three distillation steps of different temperature as described above. In another embodiment, the invention concerns the use of the fraction as described above for the manufacture of agriculturally or pharmaceutically active compounds or intermediates of agriculturally or pharmaceutically active compounds. Such use is described, for example, in WO2011/3860, WO2010037688, WO2010037688, WO2012/25469, WO2006/64251and WO2005/42468, which are all incorporated by reference in their entirety.

The invention further concerns a process for the manufacture of agriculturally or pharmaceutically active compounds or intermediates of agriculturally or pharmaceutically active compounds, comprising the distillation process according to the present invention and optionally one or more further process steps to convert the intermediates of agriculturally or pharmaceutically active compounds into agriculturally or pharmaceutically active compounds. Such processes are described, for example, in WO2011/3860, WO2010037688, WO2010037688, WO2012/25469, WO2006/64251 and WO2005/42468, which are all incorporated by reference in their entirety.

In a very preferred embodiment according to the present invention, the crude fraction of the compound of the formula (I) R1-C(O)X, wherein (I) is CDFAC, is obtained by photooxidation of 1-chloro-1,1-difluoro-2,2-dichloroethane (R 122) in the presence of O₂ and Cl₂. Preferably, the photo source is a Hg high pressure lamp, which often has 1 kw. Preferably, a glass photoreactor is held at a temperature of from 75° C. to 95° C., preferably of from 80° C. to 88° C. O₂ and Cl₂ are fed to a premixer. R 122 is pre-heated in a vaporizer to a temperature of from 70° C. to 120° C., preferably of from 72° C. to 78° C., and fed to the premixer. The premixer contents are then injected to the photoreactor. The molar ratio of O₂, in relation to R-122, preferably is from 0.35 to 0.5, in particular 0.39 to 0.45. The molar ratio of Cl₂, in relation to R-122, preferably is from 0.08 to 013, in particular 0.1 to 0.13.

Should the disclosure of any patents, patent applications, and publications which are incorporated herein by reference conflict with the description of the present application to the extent that it may render a term unclear, the present description shall take precedence.

The following examples, are intended to explain the invention further without the intent to limit it.

### EXAMPLE 1

### Manufacture of CDFAC

A Duran 50 photoreactor (volume 6 liter) was heated to 85° C., and irradiated with a 1 kw Hg high pressure lamp. O₂ was fed to a premixer at a rate of 113 g/h, and Cl₂ was fed to the premixer at a rate of 64 g/h. 1-chloro-1,1-difluoro-2,2-dichloroethane (R 122) was heated is a vaporizer to 74° C., and the evaporated R 122 was injected to the premixer. The premixer contents were then injected to the photoreactor. The molar ratio of O₂ to R 122 was 0.44, the molar ration of Cl₂ to R 122 was 0.11. The crude fraction containing CDFAC and byproducts (mainly COF₂, HCl, Cl₂, 122, COCl₂ and R 122a) was continuously drawn off the photoreactor and used further in the distillation process.

94.3% of the R 122 was reacted in the process. The selectivity was 97,06% CDFAC, 1.28% COF₂, 1.59% COCl₂ and 0.08% R 112a. The reaction was monitored and analyzed by GC (gas chromatography) and GC-MS (gas chromatography-mass spectrometry).

The photooxidation is operated continuously.

### EXAMPLE 2

### Distillation of CDFAC

The crude fraction containing CDFAC and impurities obtained by example 1 is fed to a first glass distillation column at 1 bar, which is operated at a head temperature of −20° C. A fraction containing mainly COF₂, HCl and Cl₂ is withdrawn at the top of the column and fed to a scrubber. The bottom fraction of the first distillation column is fed to a second glass distillation column at 1 bar, which is operated at a head temperature of −10° C. The bottom product of the second distillation column is a fraction containing mainly R 122 and R 112a and is fed to recycling processes. The top product of the second distillation column is fed to a third glass distillation column at 1 bar, which is operated at a head temperature of 0° C. A fraction containing mainly COCl₂ and CDFAC is withdrawn at the top of the column and fed the pre-mixer or the photoreactor of example 1, where the COCl₂ is further oxidized to CO₂, and the CDFAC is accumulated in downstream distillation steps. The bottom fraction of the third, high temperature (0° C.) distillation step is a CDFAC fraction of 99% purity by GC. The distillation is operated continuously.

1. A process for the production of a compound of the formula (I) R1-C(O)X having a reduced content of impurities, wherein R1 is CF₂H, CF₃ or CClF2 and X is a halogen, which process comprises a) subjecting a crude fraction comprising compound of formula (I) R1-C(O)X and impurities to at least two distillation steps, wherein the at least two distillation steps are performed at different temperatures and b) recovering at least a fraction of the compound of the formula (I) having a reduced content of impurities.

2. The process according to claim 1, wherein R1 is CClF2 and X is chlorine.

3. The process according to claim 1, wherein a) comprises at least three distillation steps, which consist of a low temperature distillation step, a medium temperature distillation step and a high temperature distillation step.

4. The process according to claim 3, wherein the medium temperature distillation step is carried out at a head temperature of at least 5° C. lower than the high temperature distillation step, and the low temperature distillation step is carried out at a head temperature of at least 5° C. lower than the medium temperature distillation step.

5. The process according to claim 3, wherein the head temperature difference between the high temperature distillation step and the medium temperature distillation step is from 0.5 to 15° C., and wherein the head temperature difference between the medium temperature distillation step and the low temperature distillation step is from 0.5 to 15° C.

6. The process according to claim 3, wherein the low temperature distillation step is performed first, the medium temperature distillation step is performed second and the high temperature distillation step is performed third.

7. The process according to claim 3, wherein the fraction of the compound of formula (I) having a reduced content of impurities is recovered from the high temperature distillation step as bottom product.

8. The process according to claim 1, wherein the process for the production of a compound of the formula (I) R1-C(O)X having a reduced content of impurities is carried out at a pressure of from 0.7 to 1.3 bar.

9. The process according to claim 1, further comprising a process wherein the crude fraction of the compound of formula (I) is obtained by an oxidation process starting from a compound of formula (II) R1-CHX′₂, wherein X′ is the same or different, wherein X′ is a halogen selected from the group consisting of Cl, F and Br, and wherein R1 has the same definition as above.

10. The process according to claim 3, wherein a fraction comprising the compound of formula (I) and at least one impurity is recovered from the high temperature distillation step as top product, and wherein the fraction comprising the compound of formula (I) and at least one impurity is fed into a step of the process for the manufacture of the crude fraction of the compound of formula (I) which is fed to the process for the production of a compound of the formula (I) R1-C(O)X having a reduced content of impurities.

11. The process according to claim 1, wherein the process for the production of a compound of the formula (I) R1-C(O)X having a reduced content of impurities is carried out in a distillation apparatus made or partially made or coated with or partially coated with glass or enamel.

12. The process according to claim 1, wherein the process for the production of a compound of the formula (I) R1-C(O)X having a reduced content of impurities is carried out as a continuous process.

13. A fraction of a compound of formula (I) R1-C(O)X having a reduced content of impurities, obtainable obtained by the process according to claim 1.

14. A process for the manufacture of agriculturally or pharmaceutically active compounds or intermediates of agriculturally or pharmaceutically active compounds, comprising the process according claim 1, and optionally one or more further process steps to convert the intermediates of agriculturally or pharmaceutically active compounds into agriculturally or pharmaceutically active compounds.

15. (canceled)

16. The process according to claim 9, wherein X′ is Cl.

17. The process according to claim 9, wherein a fraction comprising the compound of formula (I) and at least one impurity is recovered from the high temperature distillation step as top product, and wherein the fraction comprising the compound of formula (I) and at least one impurity is fed into a step of the process for the manufacture of the crude fraction of the compound of formula (I) which is fed to the process for the production of a compound of the formula (I) R1-C(O)X having a reduced content of impurities.
